(19) United States
(12) Patent Application Publication
(10) Pub. No.: US 2006/0200112 A1
(43) Pub. Date: Sep. 7, 2006

(54) Medical infusion device having a refillable reservoir and switch for controlling fluid direction

(76) Inventor: Patrick J. Paul, Downingtown, PA (US)

Correspondence Address: RATNERPRESTIA, P O BOX 980, VALLEY FORGE, PA, 19482-0980, US

(21) Appl. No.: 11/363,016

(22) Filed: Feb. 27, 2006

Related U.S. Application Data
(60) Provisional application No. 60/657,538, filed on Mar. 1, 2005.

(51) Int. Cl. A61K 9/22

(52) U.S. Cl. 604/890.1

(57) ABSTRACT

A device for use with an infusion pump is provided. In one exemplary embodiment the device comprises a body portion defining an orifice extending along a first axis, and a first plurality of channels extending along a second axis; and a controller disposed within the orifice and adapted to rotate within the orifice, the controller defining second plurality of channels capable of a fluid tight relationship with the first plurality of channels.

### CROSS-REFERENCE TO RELATED APPLICATIONS

This application claims the benefit of U.S. Provisional Application No. 60/657,538, filed Mar. 1, 2005, the contents of which are incorporated herein by reference.

### FIELD OF THE INVENTION

This invention relates to medical infusion devices, and more particularly to device for controlling the direction of fluid into and out of a medical infusion device.

### BACKGROUND

FIG. 1 illustrates a conventional insulin infusion pump 100. As shown in FIG. 1, insulin pump 100 consists of a control unit 102 and a disposable unit 104. Disposable unit 104 is intended to be refilled with insulin a certain number of times over the useful life of disposable unit 104. This useful life is typically 30 to 45 days, while the refill interval is typically 3 days. Consequently, over its useful life it is possible for the disposable module to be refilled 10 to 15 times or more.

FIG. 2 illustrates a block diagram of conventional disposable unit 104. As shown in FIG. 2, disposable unit 104 has a fill/refill port 202, insulin reservoir 204, filter 206, MEMS pump 208 and output port 210. Filter 206 is disposed between insulin reservoir 204 and MEMS pump 208 and coupled thereto via fluid channels 212 providing one-way filtering of insulin to MEMS pump 208.

Refilling Tools

The insulin is supplied in standard 10 ml vials. As shown in FIG. 3, by means of an adaptor 302 the insulin can be transferred to reservoir 204 (not shown in this figure) of disposable unit 104. The insulin concentration for use with the pump is typically 100 UI/ml. The use of higher or lower insulin concentrations (50, 200, 400 or 500 UI/ml) will be considered at a later stage.

In the above implementation, during the refill operation, the infusion set (not shown) is first disconnected from infusion set port 308 of disposable module 104, then, using a special adaptor 302, insulin vial 304 is attached to the disposable module 104, and finally, syringe 306 is used to create a vacuum around flexible insulin reservoir 204, to expand it, and to draw insulin into reservoir 204. It should be noted that the aforementioned disconnection from infusion port 308 is not required for a refill operation because a separate refill port 310 is provided. This approach, however, may lead to an unsafe condition if the patient decides to remain connected to infusion port 308 during a refill operation.

This particular implementation requires three different ports on the disposable module: 1) infusion set port 308; 2) insulin vial port 310; and 3) syringe port 312. In addition to the number of ports, which complicates the design of the disposable module and which makes the contamination of insulin more likely, it is impossible to ascertain, with this conventional design, the refill level of the reservoir. Furthermore, the conventional system is unable to detect whether air bubbles are injected into the reservoir during the refill operation.

From a usability standpoint, each port presents some form of surface discontinuity which needs to be carefully managed with caps, covers and/or other protection to ensure that they do not present the potential to create discomfort for the patient.

### SUMMARY OF THE INVENTION

According to one aspect of the present invention a device for simplifying the refill process of a medical infusion device is provided.

According to another aspect of the present invention, the device comprises a body portion defining an orifice extending along a first axis, and a first plurality of channels extending along a second axis; and a director disposed within the orifice and adapted to rotate within the orifice, the director defining second plurality of channels capable of a fluid tight relationship with the first plurality of channels.

According to a further aspect of the present invention, a refill level of the medical infusion device is ascertained.

According to yet another aspect of the present invention, the exemplary device monitors and detects the presence of air bubbles in the refill fluid.

According to still another aspect of the present invention, the exemplary device filters the refill fluid before the fluid enters the reservoir of the medical infusion device.

According to yet a further aspect of the present invention, a system for use with a source of fluid to provide the fluid to a user via an infusion set is provided. The system comprises an input/output port; a pump element having an input and an output; a reservoir to store a quantity of the fluid; and a director coupled to the input and output of pump element, the reservoir, and input/output port, the director adapted to direct the flow of the fluid i) from the source of fluid via input/output port and into the reservoir via the pump element when the controller is in a first position, and ii) from the reservoir and to the user via the input/output port when the controller is in a second position.

These an other aspects will become apparent in view of the detailed description provided below.

### BRIEF DESCRIPTION OF THE DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following Figures:

FIG. 1 is a perspective view of a conventional insulin infusion pump;

FIG. 2 is a block diagram of a disposable portion of the insulin pump of FIG. 1;

FIG. 3 is a perspective view of a refill operation of the insulin pump of FIG. 1;

FIGS. 4A-4C illustrate fluid flow diagrams according to an exemplary embodiment of the present invention;

FIG. 5A is a top perspective view of an exemplary embodiment of the present invention with the directional control in a first position;

FIG. 5B is a top perspective view of the exemplary embodiment of FIG. 5A with the directional control in a second position;

FIG. 6 is a side view of an exemplary fluid control switch (controller) of the present invention;

FIG. 7 is a bottom perspective view of the exemplary embodiment of FIG. 5A;

FIG. 8 is a perspective view of an exemplary receiver (compression ring) for the fluid control switch of FIG. 6;

FIG. 9 is a transparency view of the exemplary receiver of FIG. 8;

FIG. 10 is a rear perspective view of the fluid control switch of FIG. 6 in mating relation with the receiver of FIG. 8;

FIGS. 11A-11B are cross-sectional views of the exemplary embodiment of FIG. 5A;

FIG. 12 is an enlarged transparency top view of a portion of the exemplary embodiment of FIG. 5A illustrating fluid flow with the directional control in the first position; and

FIG. 13 is an transparency enlarged top view of a portion of the exemplary embodiment of FIG. 5B illustrating fluid flow with the directional control in the second position.

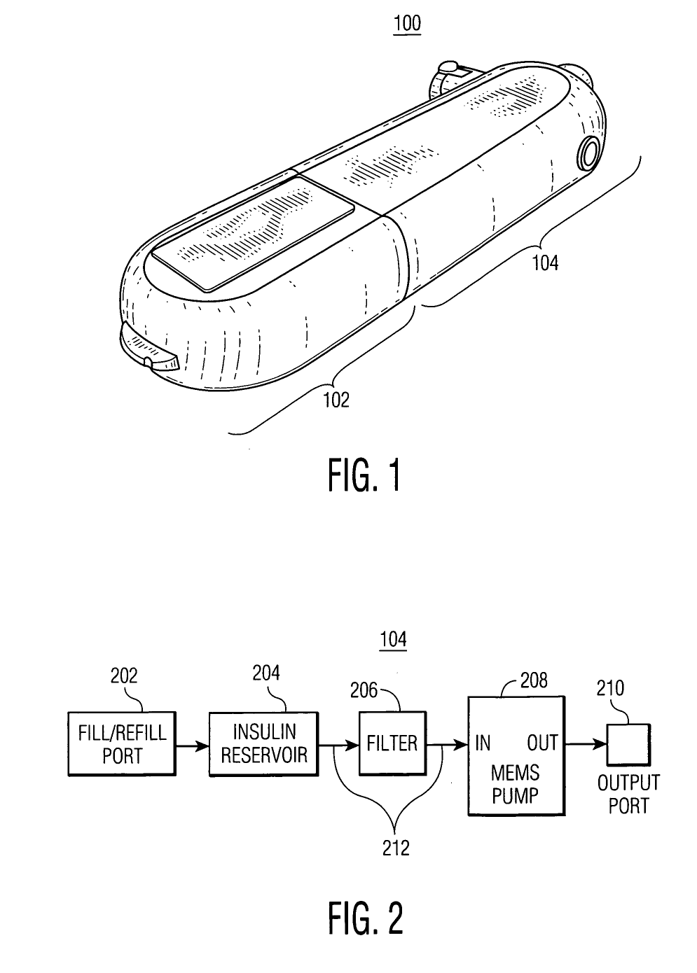

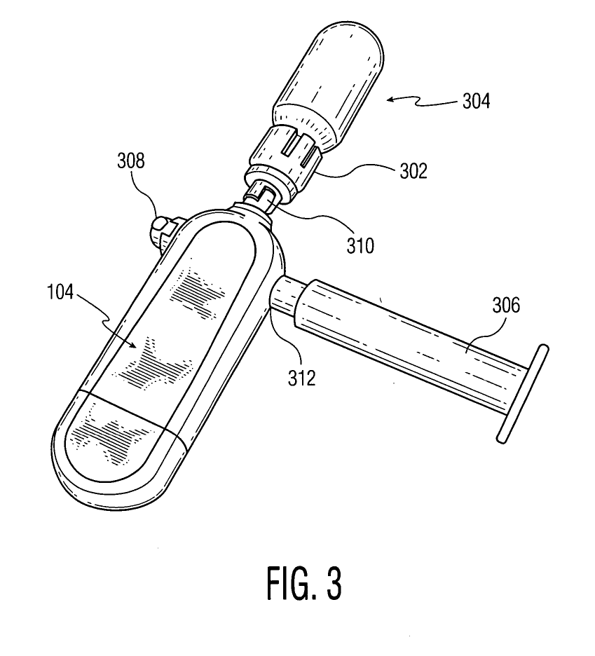

### DETAILED DESCRIPTION OF THE INVENTION

The following describes an exemplary device and method to fill and refill the insulin reservoir of a disposable insulin pump. Although the exemplary embodiments described below are illustrated in the context of a MEMS chip pump for the fill/refill operation, the invention is not so limited. It is also contemplated that the present invention may be used in conjunction with other types of pumps, such as a micro-peristaltic pump for example.

The present invention is based on the implementation of a fluidic switch to allow a change in the direction of the insulin flow depending on whether the pump is in delivery/infusion mode or in fill/refill mode.

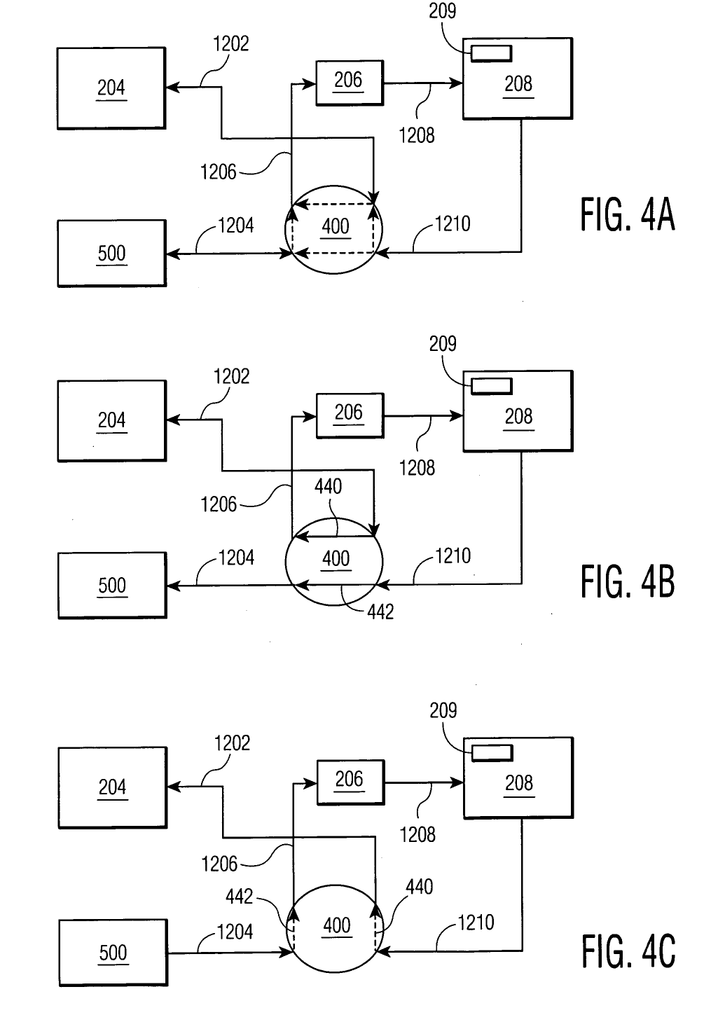

FIGS. 4A-4C are block diagrams illustrating fluid flow within an exemplary embodiment of the present invention. As shown in FIG. 4A, fluid channels 1202-1210 route fluid to and from the different components as follows:

1) to/from insulin reservoir 204 via fluid channel 1202;

2) to fluid inlet port of MEMS chip micro-pump 208 via fluid channel 1208, and from fluid outlet port of MEMS pump 208 via fluid channel 1210. MEMS pump 208 may optionally include on-chip secondary filter 209;

3) to a primary filter 206 via fluid channel 1206 to desirably remove particles from the insulin during both fill and dispense operations to ensure that there are no detrimental effects on the operation of the MEMS-chip (such as damaged or stuck valves, for example);

4) to/from a combined fluid port connection 500 for connection to i) an infusion set (not shown) when the exemplary control circuit is in a first position (described below) or ii) a source of insulin, such as an insulin refill vial (not shown in this figure) when the exemplary control circuit is in a second position (also described below), via fluid channel 1204;

5) to/from a fluidic selection switch 400 tasked to route fluid paths 440, 442 (best shown in FIG. 8—illustrated as dashed lines in FIG. 4A) based on its position.

Referring now to FIG. 4B, the fluid delivery mode of an exemplary embodiment of the present invention is illustrated. As shown in FIG. 4B, in the delivery mode, the two (2) fluid channels 440, 442 within fluidic switch 400 are positioned such that one of the fluid channels is coupled between insulin reservoir 204 and the input of MEMS pump 208 (or optionally the input of filter 206), while the other fluid channel is coupled between the output of MEMS pump 208 and input/output port 500 and ultimately to the infusion set (not shown). In the non-limiting example shown, fluid channel 440 connects fluid channels 1202 and 1206, and fluid channel 442 connects fluid channels 1204 and 1210.

In operation, the insulin flow is from reservoir 204, flows through fluid channel 440, into primary filter 206 and then into the inlet port of MEMS micro-pump 208. Under the pumping action of the MEMS micro-pump 208, the fluid is expelled from MEMS pump outlet port, and then routed to the fluid connection port 500 (end infusion set) via the fluid channel 442.

Referring now to FIG. 4C, the fluid fill/refill mode of an exemplary embodiment of the present invention is illustrated. In this configuration, the fluid (insulin) is drawn from the insulin vial (not shown in this figure) which is attached to the same fluid connection port 500 used for infusion. Insulin then flows through fluid channel 442 of fluidic switch 400 and is then routed to primary filter 206 and enters the MEMS micro-pump 208 via the inlet port. In the non-limiting example shown, fluid channel 440 connects fluid channels 1202 and 1210, and fluid channel 442 connects fluid channels 1204 and 1206. The insulin is then expelled by the MEMS pump 208 via its outlet port and is ultimately routed to the reservoir 204 via fluid channel 440 of fluidic switch 400.

Accordingly, primary filter 206 may thus used to eliminate the presence of particles in suspension in the insulin both when reservoir 204 is filled and when reservoir 204 is depleted.

In summary, MEMS pump 208 can be used to both draw insulin from the disposable reservoir for the purpose of dispensing this insulin to the patient or draw insulin from the insulin vial to fill/refill the reservoir of the disposable module. To this end, a two section fluid switch is used to implement this directable flow of insulin.

In use, a pressure sensor (not shown) associated with pump 208, which may be either internal or external to pump 208, may be used to detect a high pressure condition during either or both the infusion mode and/or fill/re-fill mode. The high pressure condition in the fill/re-fill mode is indicative of either a reservoir full condition, an air in reservoir condition, or other flow restriction. In the infusion mode, a high pressure condition is indicative of an occlusion in the system.

Fluidic Switch Implementation

The novel fluidic switch can be implemented in a number of ways. Although the description hereafter provided illustrates one exemplary embodiment of the present invention, the invention is not so limited in that it may be carried out using alternative approaches such as cam systems, pinching or releasing tubing, etc. Accordingly, these equivalent approaches are considered to be part of the present invention.

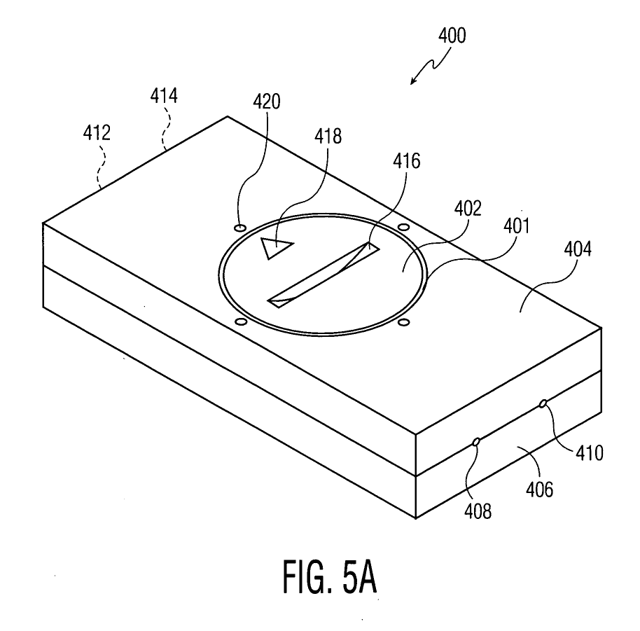

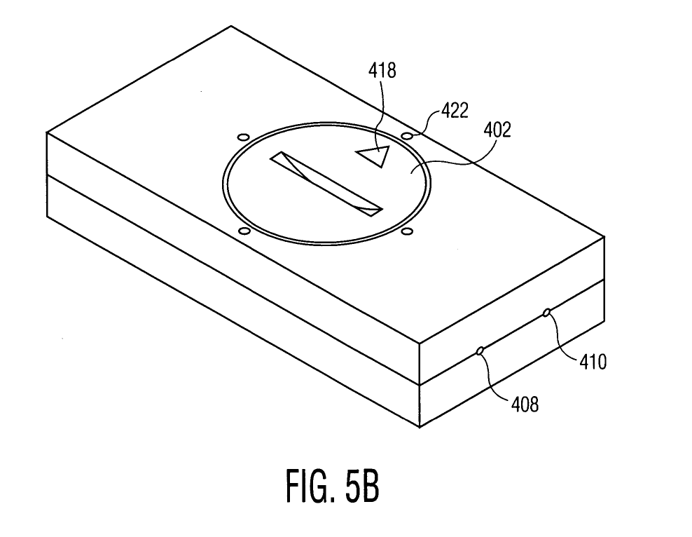

FIGS. 5A-5B illustrate top perspective views of an exemplary embodiment of the present invention. As shown in FIG. 5A, fluidic switch 400 comprises an upper body portion 404 and a lower body portion 406 coupled to one another at opposing faces. Upper and lower body portions 404 and 406 define a receiver portion 401 into which director 402 may be rotatably coupled. Body portions 404 and/or 406 also define fluid channels 408, 410, 412 and 414 which, in the non-limiting illustration, extend along an axis of fluid switch 400 and into which other components of the overall system, such as a pump, reservoir and/or I/O ports may be coupled as desired.

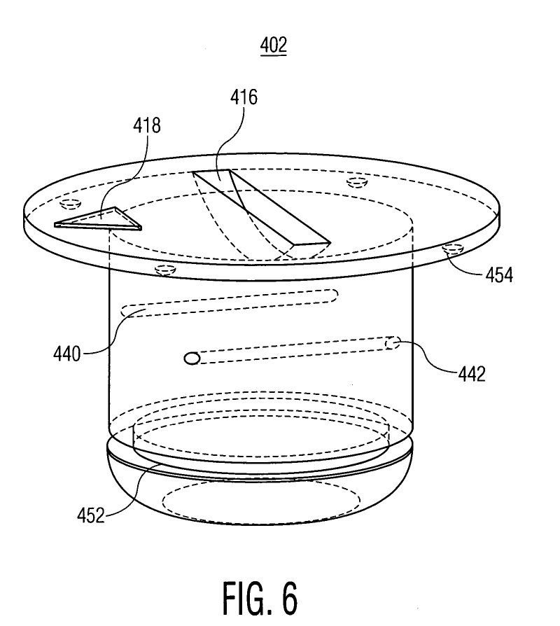

In one exemplary embodiment of the present invention (best shown in FIG. 6), director 402 is in the form of a cylindrical bushing. This bushing is desirably molded from an insulin-compatible material, such as polycarbonate, and comprises two channels 440, 442 within its body.

In one exemplary embodiment, the top surface of director 402 has a disc-like shape and defines a “coin slot” 416, or other means for repositioning director 402, and a visual indicator 418 (in this case a chevron shape), which may be aligned with indicators, such as 420, to allow the user to easily change the position of director 402 and readily determine the position of director 402. Further, means to positively align director 402 with body portions 404, 406 may be provided, such as with dimples disposed on an underside of the upper surface of director 402 and corresponding depressions formed on an upper surface of body portion 404 onto which director 402 interfaces, for example.

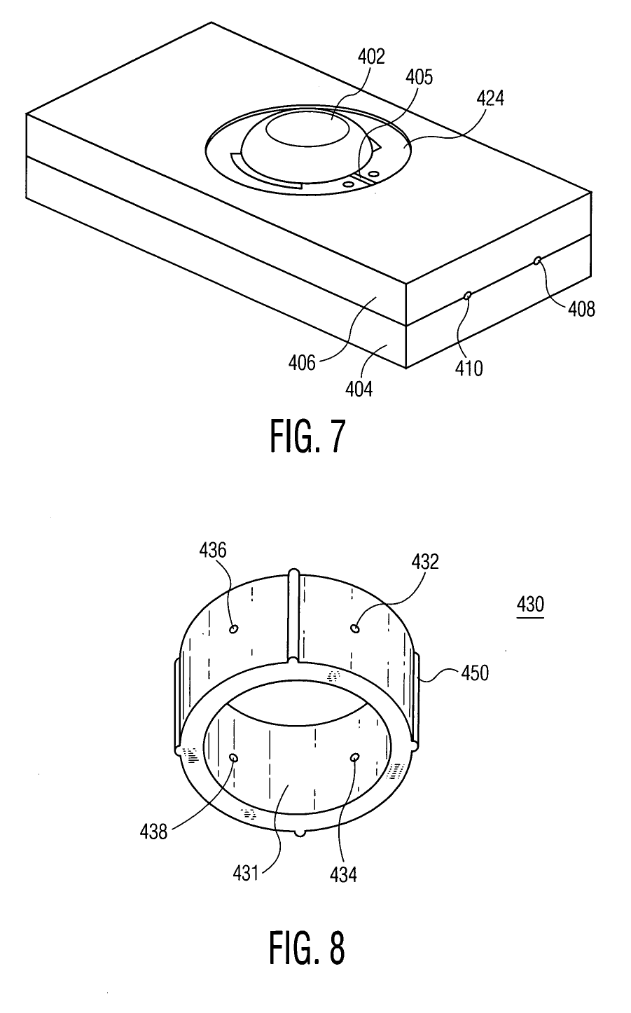

Referring again to FIG. 6, the base of the director 402 defines a circumferential groove 452 adapted to receive a retainer, such as a well-known retaining clip 424 (best shown in FIG. 7), which is used in this particular implementation to maintain director 402 within the fluidic switch assembly 400. When in position within the body of the disposable module, the top surface of the director 402 is visible through the housing of the module and the “coin slot” 416 is accessible to the patient to allow for director 402 to be rotated in either the “infusion” position, or in the “fill/refill” position. To switch from one position to the other, merely requires rotation of director 402 90 degrees in either direction.

In one exemplary embodiment, when installed within the disposable module, director 402 rotates within an elastomer ring 430 (best shown in FIG. 8). The compression of this elastomer by director 402 provides for hermeticity of the different fluid paths under any rotational position of the director 402. The presence of the elastomer also provides for a hermetic seal against the ingress of fluid from the outside of the pump into the disposable module or into the fluid passages.

FIG. 7, illustrates a bottom perspective view of an exemplary embodiment of the present invention in which the relationship between director 402, body portion 406 and retainer 424 is shown. An exemplary retainer 424 may be a well-know “E” clip that is matingly coupled to groove 452 (best shown in FIG. 6) and rests in seat 405 defined in a lower surface of body portion 406. The “spring-action” of retainer 424 can also be viewed as part of a default mechanism to prevent any free-rotation of director 402.

FIG. 8 illustrates a 3-D rendition of an elastomer compression-ring 430 within which director 402 is disposed. As shown in FIG. 8, compression ring 430 defines an orifice 431 to receive director 402. Compression ring 430 also comprises at least one member 450 (in this embodiment ribs) which mates with complementary grooves in body portions 404 and/or 406 (not shown in this figure), so as to prevent compression ring 430 from rotating within body portions 404, 406, thus maintaining proper alignment.

Compression ring 430 also defines thru passages 432, 434, 436, 438 which provide for bi-directional fluid passage from the inner surface of compression ring 430 to the outer surface of compression ring 430. Desirably, the elastomer used for compression ring 430 is compressible and made of a chemically neutral material, such as silicone for example. The purpose of this compression ring is to provide a hermetic seal (air and water/insulin) between director 402 and body portions 404/406 of fluidic switch 400.

In one exemplary embodiment, compression ring 430 is located immediately under the top surface of director 402 and provides a seal against ingress of liquid or other contaminants from the outside of the pump. Additionally, to prevent/neutralize the ingress of any contaminant, it is contemplated that compression ring 430 could also be impregnated with an anti-bacterial agent.

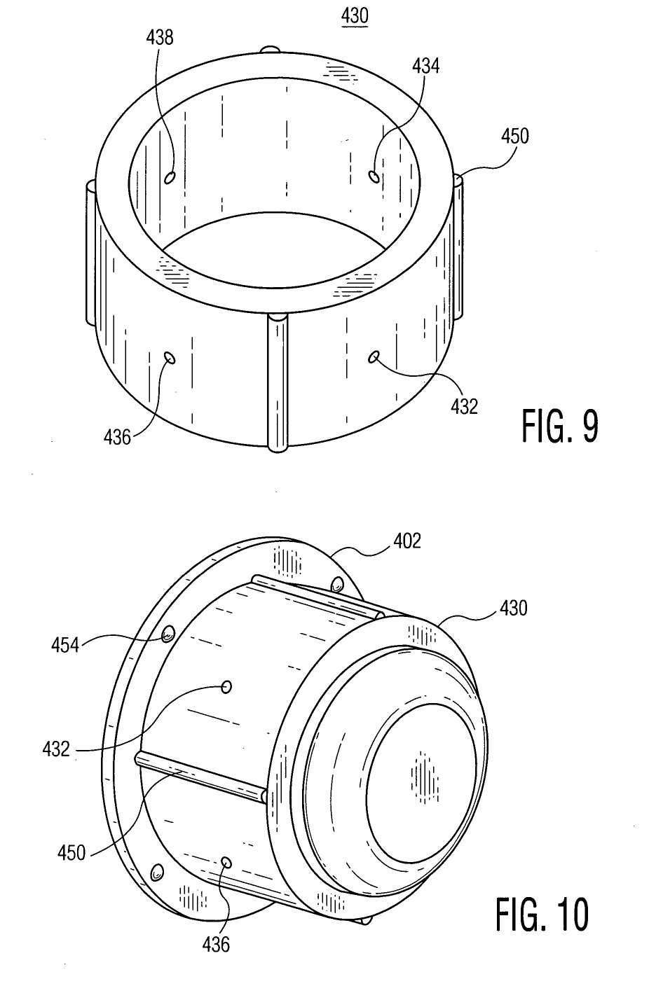

FIG. 9 illustrates a CAD rendition of elastomeric compression ring 430, this time in a “transparent” configuration. The purpose of this illustration is to better show fluid passages 432, 434, 436, 438 between the inner and the outer surfaces of compression ring 430.

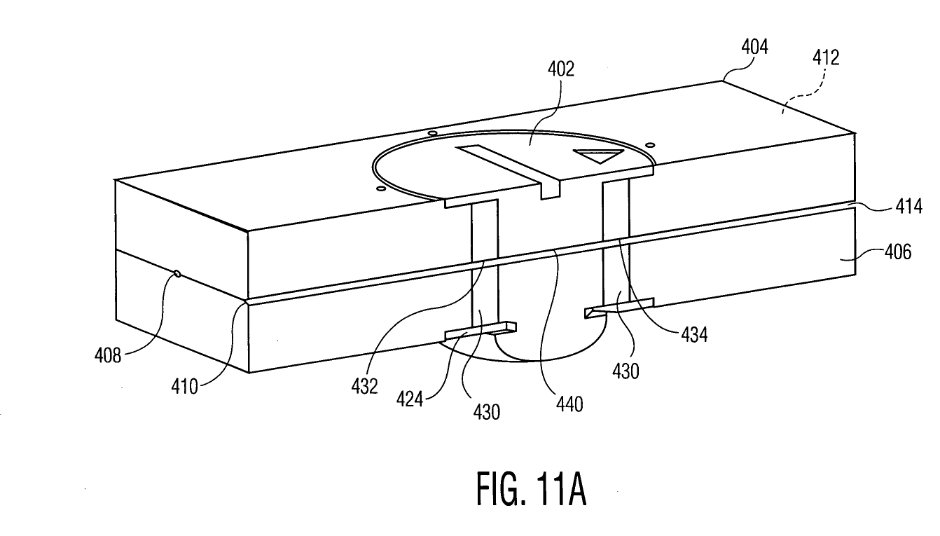

FIG. 10 illustrates director 402 disposed within elastomer compression ring 430. As is evident from FIG. 10, director 402 is free to rotate within elastomer ring 430, with elastomer ring 430 providing a predetermined amount of compression to maintain a fluid tight seal between ring 430 and director 402. Typically, the inside diameter of compression ring 430 would be a few percent smaller then the outside diameter of director 402 so that a slight expansion of the ring would result when director 402 is inserted in compression ring 430, and mutual friction on seal would result.

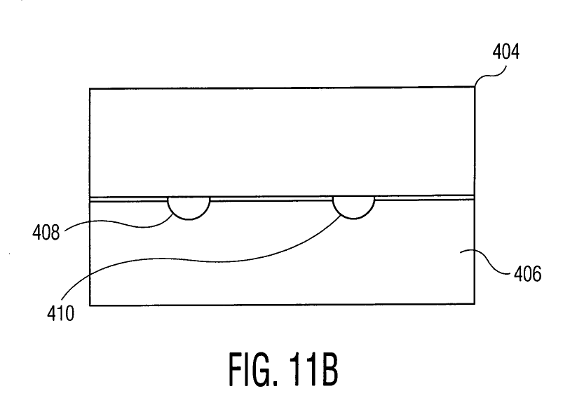

FIG. 11A, illustrates the relationship between body portions 404, 406, director 402 and compression ring 430 according to one exemplary embodiment of the present invention. As shown in FIG. 11A, compression ring 430 is disposed within body portions 404, 406 of fluidic switch 400. Director 402 is in turn disposed within compression ring 430.

FIGS. 11A-11B also show fluid channels 408, 410, 412, 414 defined by housings 402 and/or 404, fluid channels 432, 434 defined by compression ring 430 and fluid channel 440 defined by director 402, as well as the relationship between these various channels. With respect to fluid channels 408, 410, 412, 414, in one exemplary embodiment these fluid channels are formed during molding of one of body portions 404, 406 with a groove. The grooved body portion may then be bonded to the other body portion. It is also possible to mold each of body portions 404, 406 with matching grooves, if desired, although such an approach may complicate the assembly process. The material that may be used to form body portions is desirably a plastic although the invention is not so limited in that other materials and process may be used to form body portions 404, 406. When the two body portions 404, 406 of the housing are bonded together, grooves 408, 410, 412, 414 now becomes fully enclosed and forms the desired fluid channels.

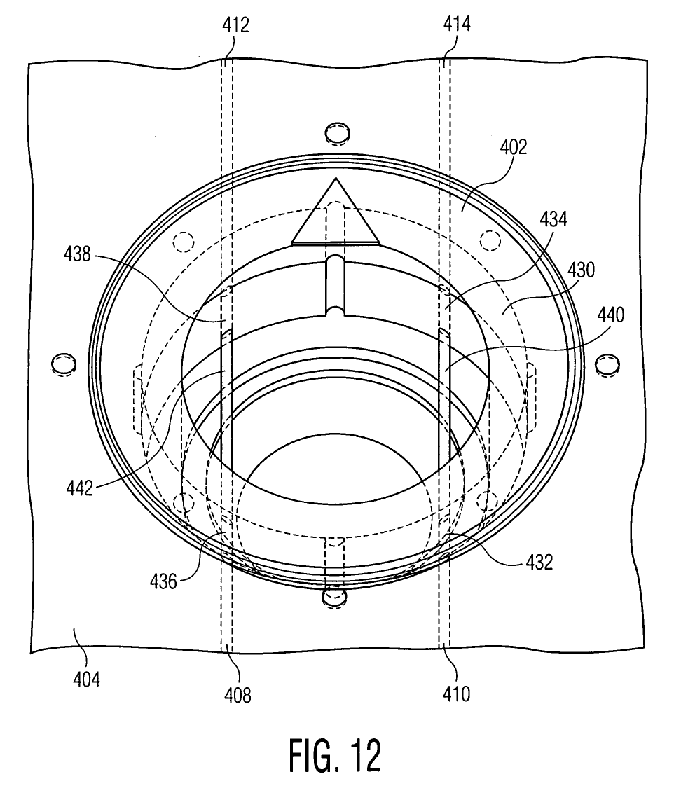

FIG. 12 illustrates an enlarged top view of director 402, compression ring 430 and housing portion 404, with director 402 rotated to a first position. As shown is FIG. 12, in the illustrated position, fluid is allowed to pass between fluid channels 408 and 412 via channels 436, 438 formed in compression ring 430 and channel 442 formed in director 402. Simultaneously, fluid is allowed to pass between fluid channels 410 and 414 via channels 432, 434 formed in compression ring 430 and channel 440 formed in director 402.

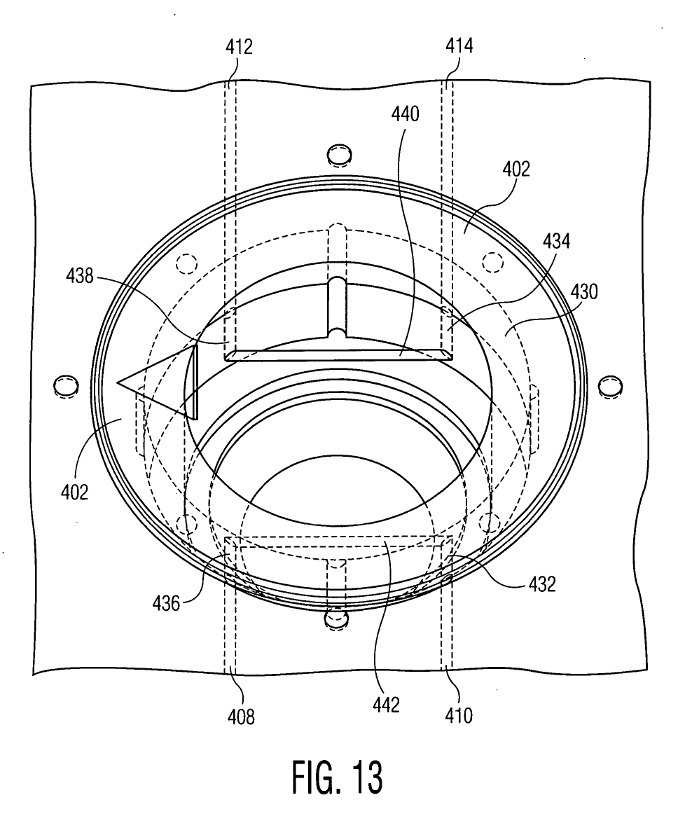

FIG. 13 illustrates an enlarged top view of director 402, compression ring 430 and housing portion 404, with director 402 rotated to a second position. In this case the second position is based on rotating director 402 90° counter-clockwise. The invention is not so limited in that an equivalent position may be attained by rotating director 402 90° clockwise. As shown is FIG. 13, in the illustrated position, fluid is allowed to pass between fluid channels 408 and 410 via channels 432, 436 formed in compression ring 430 and channel 442 formed in director 402. Simultaneously, fluid is allowed to pass between fluid channels 412 and 414 via channels 434, 438 formed in compression ring 430 and channel 440 formed in director 402. As a result of this rotation there is no longer any fluid passage between fluid channels 408/412 and 410/414.

In other exemplary embodiment, the fluidic switch can also comprise an electrical switch (not shown) to provide the angular position of director 402 (confirmation of selected fluid path via and electrical signal). This switch can be used to confirm to the pump hardware/firmware that the fluidic switch has been set to the proper position before initiating a certain operation. Conversely, a change in the condition of this switch (from FILL to IN FUSE or IN FUSE to FILL, for example) may also be used to interrupt the pump processor(s) and initiate the mode change.

In another exemplary embodiment, the well-known display and keypad of a medical infusion device (not shown) may be used to preset the amount liquid medication for transfer from medication container 304, for example, to reservoir 204 of the medical infusion device

In yet another exemplary embodiment, the well-known audio indicator of a medical infusion device may be used to signal the user that the preset amount of medication has been transferred from medication container 304 to reservoir 204.

Additionally, it is contemplated that a wireless communication capability may be included in the exemplary device to signal the user that the preset amount of medication was transferred from medication container 304 to reservoir 204.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A device for use with an infusion pump, the device comprising:
a body portion defining an orifice extending along a first axis, and a first plurality of channels extending along a second axis; and
a director disposed within the orifice and adapted to rotate within the orifice, the director defining second plurality of channels capable of a fluid tight relationship with the first plurality of channels.

2. The device of claim 1, wherein the director is rotatable between at least two positions.

3. The device of claim 2, wherein:
when the director is in a first one of the positions i) a first and second of the first plurality of channels are coupled to one another via a first one of the second plurality of channels and ii) a third and fourth of the first plurality of channels are coupled to one another via a second one of the second plurality of channels, and
when the director is in a second one of the position i) the first and third of the first plurality of channels are coupled to one another via one of the second plurality of channels and ii) the second and fourth of the first plurality of channels are coupled to one another via the other of the second plurality of channels.

4. The device of claim 1, wherein the body portion is comprised of an upper portion and a lower portion, at least one of the upper and/or lower portions having the first plurality of channels formed in a surface thereof.

5. The device of claim 1, further comprising a seal disposed between an outer surface of the director and the orifice, the seal having a respect number of channels adapted to interface with the first plurality of channels in the body portion and the second plurality of channels in the director.

6. The device of claim 5, wherein the seal is resilient.

7. The device of claim 5, wherein the seal comprises an anti-bacterial agent.

8. The device of claim 1, wherein the director comprises means for indicating a position of director with respect to the body portion.

9. A system for use with a source of fluid to provide the fluid to a user via an infusion set, the system comprising:
an input/output port;
a pump element having an input and an output;
a reservoir to store a quantity of the fluid; and
a director coupled to the input and output of pump element, the reservoir, and input/output port, the director adapted to direct the flow of the fluid i) from the source of fluid via input/output port and into the reservoir via the pump element when the controller is in a first position, and ii) from the reservoir and to the user via the input/output port when the controller is in a second position.

10. The system of claim 9, further comprising a filter disposed between the director and the input of the pump element to filter the fluid i) from the source of fluid when the director is in the first position and ii) from the reservoir when the director is in the second position.

11. The system of claim 10, wherein the filter is used to filter the fluid both when the reservoir is filled with the liquid, and when the fluid is pumped out of the reservoir for infusion into the patient.

12. The system of claim 9, wherein the pump element further comprises a pressure sensor adapted to detect high fluid pressure when the fluid is infused and when the reservoir is refilled.

13. The system of claim 9, further comprising a display and a keypad adapted to preset an amount of the fluid to transfer from the source of fluid to the reservoir.

14. The system of claim 9, further comprising an audio indicator adapted to signal the user that the preset amount of medication has been transferred from the source of fluid to the reservoir.

15. The system of claim 9, further comprising a wireless communication to signal to the user that the preset amount of fluid has been transferred from the source of fluid to the reservoir.

16. The system of claim 9, wherein an operating mode of the system changes based on modifying the position of the director.

17. A medical infusion device for use with a liquid medication, the infusion device comprising:
a pump;
a reservoir coupled to the pump; and
a single fluid port,
wherein in a first mode the fluid port is used to substantially fill the reservoir with the liquid medication, and in a second mode the fluid port if used to dispense the liquid medication to a patient.
